(19) United States
(12) Patent Application Publication
(10) Pub. No.: US 2025/0246308 A1
(43) Pub. Date: Jul. 31, 2025

(54) SPACETIME ATTENTION FOR CLINICAL OUTCOME PREDICTION

(71) Applicant: GENENTECH, INC., South San Francisco, CA (US)

(72) Inventors: Degan HAO, South San Francisco, CA (US); Mohammadreza NEGAHDAR, South San Francisco, CA (US)

(21) Appl. No.: 19/087,315

(22) Filed: Mar. 21, 2025

Related U.S. Application Data
(63) Continuation of application No. PCT/US2023/074825, filed on Sep. 22, 2023.
(60) Provisional application No. 63/376,957, filed on Sep. 23, 2022.

(51) Int. Cl. G16H 50/20; G16H 10/60; G16H 50/80

(52) U.S. Cl. CPC G16H 50/20; G16H 10/60; G16H 50/80

(57) ABSTRACT

A disease prognosis model to may be trained to determine the clinical outcome of a disease based on longitudinal data including a health record for each timepoint in a sequence of timepoints. The disease prognosis model may include a recurrent neural network trained to extract, from each health record, a feature set representative of local dependencies present within the health record. The disease prognosis model may include a spacetime attention trained to determine an importance of each feature in the feature set at each timepoint in the sequence of timepoints. The disease prognosis model may include a feedforward neural network trained to determine, based on the importance of each feature in the feature set at each time point in the sequence of timepoints, the clinical outcome of the disease. The trained disease prognosis model may be applied to determine the clinical outcome of the disease for one or more patients.

### CROSS REFERENCE TO RELATED APPLICATION

This application claims priority to U.S. Provisional Application No. 63/376,957, entitled “SPACETIME ATTENTION FOR CLINICAL OUTCOME PREDICTION” and filed on Sep. 23, 2022, the disclosure of which is incorporated herein by reference in its entirety.

### TECHNICAL FIELD

The subject matter described herein relates generally to machine learning and more specifically to a machine learning based prognosis model for predicting the clinical outcome of a disease.

### INTRODUCTION

A number of diseases can present serious long term complications. For example, the infection of the novel coronavirus SARS-CoV-2 can cause coronavirus disease (COVID-19) with clinical syndromes including coughing, headache, fever, etc. Among the patients infected by SARS-CoV-2, a significant number will develop sustained post-infection sequelae. Patients with COVID-19 can present, for at least two months after the initial acute infection, prolonged COVID-19 symptoms such as fatigue, dyspnea, and memory problems. In some cases, the symptoms associated with COVID-19 could emerge, relapse, and linger for months and even years after the initial acute infection. Chronic COVID-19 symptoms can even be life threatening in the most severe cases. As such, identifying cohorts with high risk of severe long-term disease complications may be beneficial towards treatment planning as well as medical resource allocation.

### SUMMARY

Systems, methods, and articles of manufacture, including computer program products, are provided for machine learning enabled prediction of clinical outcome. In one aspect, there is provided a system for machine learning enabled prediction of clinical outcome. The system may include at least one processor and at least one memory. The at least one memory may include program code that provides operations when executed by the at least one processor. The operations may include: training a disease prognosis model to determine, based at least on longitudinal data, a clinical outcome of a disease, the longitudinal data including a health record for each timepoint in a sequence of timepoints, the training of the disease prognosis model includes training a recurrent neural network, a spacetime attention, and a feedforward neural network, the recurrent neural network being trained to extract, from each health record, a feature set representative of one or more local dependencies present within the health record, the spacetime attention being trained to determine an importance of each feature in the feature set at each timepoint in the sequence of timepoints, and the feedforward neural network being trained to determine, based at least on the importance of each feature in the feature set at each time point in the sequence of timepoints, the clinical outcome of the disease; and applying the trained disease prognosis model to determine, based at least on a first health record from a first timepoint and a second health record from a second timepoint, the clinical outcome of the disease for a patient associated with the first health record and the second health record.

In another aspect, there is provided a method for machine learning enabled prediction of clinical outcome. The method may include: training a disease prognosis model to determine, based at least on longitudinal data, a clinical outcome of a disease, the longitudinal data including a health record for each timepoint in a sequence of timepoints, the training of the disease prognosis model includes training a recurrent neural network, a spacetime attention, and a feedforward neural network, the recurrent neural network being trained to extract, from each health record, a feature set representative of one or more local dependencies present within the health record, the spacetime attention being trained to determine an importance of each feature in the feature set at each timepoint in the sequence of timepoints, and the feedforward neural network being trained to determine, based at least on the importance of each feature in the feature set at each time point in the sequence of timepoints, the clinical outcome of the disease; and applying the trained disease prognosis model to determine, based at least on a first health record from a first timepoint and a second health record from a second timepoint, the clinical outcome of the disease for a patient associated with the first health record and the second health record.

In another aspect, there is provided a computer program product for machine learning enabled prediction of clinical outcome. The computer program product may include a non-transitory computer readable medium storing instructions that cause operations when executed by at least one data processor. The operations may include: training a disease prognosis model to determine, based at least on longitudinal data, a clinical outcome of a disease, the longitudinal data including a health record for each timepoint in a sequence of timepoints, the training of the disease prognosis model includes training a recurrent neural network, a spacetime attention, and a feedforward neural network, the recurrent neural network being trained to extract, from each health record, a feature set representative of one or more local dependencies present within the health record, the spacetime attention being trained to determine an importance of each feature in the feature set at each timepoint in the sequence of timepoints, and the feedforward neural network being trained to determine, based at least on the importance of each feature in the feature set at each time point in the sequence of timepoints, the clinical outcome of the disease; and applying the trained disease prognosis model to determine, based at least on a first health record from a first timepoint and a second health record from a second timepoint, the clinical outcome of the disease for a patient associated with the first health record and the second health record.

In some variations of the methods, systems, and non-transitory computer readable media, one or more of the following features can optionally be included in any feasible combination.

In some variations, the recurrent neural network may be a bi-directional recurrent neural network (RNN), a long short-term memory (LSTM) network, a local long short-term memory (LSTM) network with a given window size for timepoints, or a gated recursive unit (GRU) network.

In some variations, the feedforward neural network may be a multi-layer perceptron model.

In some variations, the trained disease prognosis model may determine the clinical outcome of the disease for the patient associated with the first health record and the second health record by at least applying the trained recurrent neural network to extract, from the first health record, a first set of feature values for a hidden feature set representative of a first set of local dependencies present within the first health record, and applying the trained recurrent neural network to extract, from the second health record, a second set of feature values for the hidden feature set representative of a second set of local dependencies present within the second health record

In some variations, the trained recurrent neural network may output, for ingestion by the trained spacetime attention, a feature map comprising the first set of feature values from the first timepoint and the second set of feature values from the second timepoint.

In some variations, the trained disease prognosis model may further determine the clinical outcome of the disease for the patient associated with the first health record and the second health record by at least applying the trained spacetime attention to determine, based at least on a feature map including the first set of feature values and the second set of feature values, the importance of each feature in the hidden feature set at each of the first timepoint and the second timepoint.

In some variations, the trained spacetime attention may include one or more two-dimensional convolutional layers trained to determine the importance of each feature in the hidden feature set across a time dimension and a feature dimension.

In some variations, the one or more two-dimensional convolutional layers may include 1×1 convolutional filters configured to perform a joint weighting of the importance of each feature in the hidden feature set across the time dimension and the feature dimension.

In some variations, the trained spacetime attention may determine, for a first feature from the hidden feature set, a first importance of the first feature at the first timepoint and a second importance of the first feature at the second timepoint.

In some variations, the trained spacetime attention may further determine, for a second feature from the hidden feature set, a third importance of the second feature at the first timepoint and a fourth importance of the second feature at the second timepoint.

In some variations, the trained disease prognosis model may further determine the clinical outcome of the disease for the patient associated with the first health record and the second health record by at least applying the trained feedforward neural network to determine, based at least on the importance of each feature in the hidden feature set at each of the first timepoint and the second timepoint, the clinical outcome of the disease for the patient.

In some variations, the disease prognosis model may be further trained to determine the clinical outcome of the disease based on non-longitudinal data. The non-longitudinal data may be static across the sequence of timepoints. The non-longitudinal data may be concatenated with the health record associated with each timepoint in the sequence of timepoints.

In some variations, one or more missing values for a non-longitudinal variable comprising the non-longitudinal data may be identified. The one or more missing values may be replaced with a mean value of the non-longitudinal variable observed in an available dataset.

In some variations, the non-longitudinal data may include medical image data and/or electrogram data corresponding to a metric quantifying a severity of the disease portrayed in one or more medical images and/or electrograms.

In some variations, the health record associated with each timepoint in the sequence of timepoints may include a value for each of a plurality of vital sign statistics.

In some variations, the health record associated with each timepoint in the sequence of timepoints may include a value for each of a plurality of laboratory test variables.

In some variations, the health record associated with each timepoint in the sequence of timepoints include medical image data and/or electrogram data. The medical image data includes a metric quantifying a severity of the disease portrayed in one or more medical images and/or electrograms.

In some variations, the longitudinal data may be determined to include a missing value for a longitudinal variable at a first timepoint. The missing value may be replaced with (i) a first value of the longitudinal variable from a second timepoint preceding the first timepoint, (ii) a second value of the longitudinal variable from a third timepoint following the first timepoint, or (iii) a third value determined based on the first value and the second value.

In some variations, the disease may be coronavirus disease (COVID-19), Alzheimer's disease, or age-related macular degeneration.

In some variations, the clinical outcome of the disease may include a probability associated with one or more of cure, worsening, and mortality.

Implementations of the current subject matter can include, but are not limited to, methods consistent with the descriptions provided herein as well as articles that comprise a tangibly embodied machine-readable medium operable to cause one or more machines (e.g., computers, etc.) to result in operations implementing one or more of the described features. Similarly, computer systems are also described that may include one or more processors and one or more memories coupled to the one or more processors. A memory, which can include a non-transitory computer-readable or machine-readable storage medium, may include, encode, store, or the like one or more programs that cause one or more processors to perform one or more of the operations described herein. Computer implemented methods consistent with one or more implementations of the current subject matter can be implemented by one or more data processors residing in a single computing system or multiple computing systems. Such multiple computing systems can be connected and can exchange data and/or commands or other instructions or the like via one or more connections, including, for example, to a connection over a network (e.g. the Internet, a wireless wide area network, a local area network, a wide area network, a wired network, or the like), via a direct connection between one or more of the multiple computing systems, etc.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes in relation to the prediction of clinical outcomes in the context of post-acute sequelae of COVID-19, it should be readily understood that such features are not intended to be limiting. The claims that follow this disclosure are intended to define the scope of the protected subject matter.

### DESCRIPTION OF THE DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 depicts a system diagram illustrating an example of a prognosis system, in accordance with some example embodiments;

FIG. 2 depicts a schematic diagram illustrating an example of a spacetime attention calculating feature importance across a time dimension as well as a feature dimension, in accordance with some example embodiments;

FIG. 3A depicts a schematic diagram illustrating an example of a disease prognosis model, in accordance with some example embodiments;

FIG. 3B depicts a schematic diagram illustrating an example of a disease prognosis model, in accordance with some example embodiments;

FIG. 4A depicts a flowchart illustrating an example of a process for training a disease prognosis model to perform clinical outcome prediction, in accordance with some example embodiments;

FIG. 4B depicts a flowchart illustrating an example of a process for machine learning enabled clinical outcome prediction, in accordance with some example embodiments;

FIG. 4C depicts a flowchart illustrating an example of a process for machine learning enabled clinical outcome prediction, in accordance with some example embodiments;

FIG. 5A depicts a graph illustrating an example of a patient's severity of disease being classified based on an Acute Physiology and Chronic Health Evaluation II (APACHE II) score over time, in accordance with some example embodiments;

FIG. 5B depicts a graph illustrating an example of a patient's Acute Physiology and Chronic Health Evaluation II (APACHE II) score being decomposed by different physiologic variables, in accordance with some example embodiments;

FIG. 5C depicts a table illustrating an example output of a spacetime attention, in accordance with some example embodiments; and

FIG. 6 depicts a block diagram illustrating an example of a computing system, in accordance with some example embodiments.

When practical, similar reference numbers denote similar structures, features, or elements.

### DETAILED DESCRIPTION

Identifying cohorts with high risk of severe long-term morbidity may benefit treatment planning as well as medical resource allocation. In the case of COVID-19, identifying patients at high risk of severe long-term complications may be essential in providing timely medical intervention. Accurate prognosis for long-term disease outcome, such as prolonged morbidity and mortality, requires an integrated assessment of multimodal data including static, non-longitudinal data, such as a patient's initial condition at the onset of disease, as well as longitudinal data tracking disease progression. Nevertheless, due to the heterogeneous phenotypes and chronic conditions presented in patients, it may be difficult to predict patient outcomes from longitudinal data such as a series of electronic health records obtained at different timepoints. In particular, longitudinal data exhibits a combination of short-term and long-term dependencies that evades conventional approaches to disease outcome prognosis. For example, in patients who have been hospitalized due to COVID-19 pneumonia, fibrotic-like abnormalities that are common during the first three months will have mostly disappeared by the one year mark whereas consolidations are usually resolved within six months. Accordingly, in the case of COVID-19 and the likelihood of prolonged and/or recurring symptoms, the feature importance of fibrosis should not only change over time but also differ from that of consolidation. However, conventional approaches to disease outcome prognosis can either recognize time-dependent feature importance in the absence of feature diversity or provide spatial feature importance that is static over time.

As such, in some example embodiments, a disease prognosis model for determining the clinical outcome of a disease may include a spacetime attention mechanism capable of weighing feature importance jointly from the temporal dimension and feature space of longitudinal data. Examples of longitudinal data include medical data in the form of a health record (e.g., an electronic health record (EHR)) for each timepoint in a sequence of successive timepoints. As used herein, the term “health record” may refer to an assortment of data spanning a variety of modalities. For example, each health record may include a value for each of a plurality of vital statistics such as systolic blood pressure, diastolic blood pressure, pulse rate, respiratory rate, and/or the like. Alternatively and/or additionally, each health record may include a value for each of a plurality of laboratory test variables. Examples of laboratory test variables may include Fibrinogen, C Reactive Protein, Pro-thrombin International Normalized Ratio, Prothrombin Time, Lactate Dehydrogenase, D-Dimer, Albumin, Ferritin, Alanine Aminotransferase, Aspartate Aminotransferase, Chloride, Protein, Alkaline Phosphatase, Bilirubin, Calcium, Creatinine, Glucose, Hematocrit, Hemoglobin, Potassium, Platelets, Erythrocytes, Sodium, Leukocytes, and/or the like. In some cases, the health record associated with each timepoint in the sequence of timepoints may include medical image data associated with X-rays, magnetic resonance imaging (MRI) scans, computed tomography (CT) scans, positron emission tomography (PET) scans, optical coherence tomography (OCT) scans, and/or the like. Furthermore, in some cases, each health record may also include one or more electrogram data associated with electroencephalogram (EEG), electrocorticography (ECoG or iEEG), electrooculogram (EOG), electroretinogram (ERG), electrocardiogram (ECG), electromyogram (EMG), and/or the like.

In some example embodiments, the disease prognosis model may include a recurrent neural network and a feedforward neural network coupled with the spacetime attention mechanism. For example, the recurrent neural network may be trained to extract, from each health record included in the longitudinal data, a feature set representative of one or more local dependencies present within the health record. The spacetime attention may be trained to determine an importance of each feature in the feature set at each timepoint in the sequence of timepoints. Moreover, the feedforward neural network may be trained to determine, based at least on the importance of each feature in the feature set at each time point in the sequence of timepoints, the clinical outcome of the disease. The combination of the recurrent neural network and the spacetime attention enables the disease prognosis model to effectively capture the short-term and long-term dependencies present within longitudinal data such as a series of health records (e.g., electronic health records) from different timepoints. In particular, the trained disease prognosis model may be applied to identify critical timepoints and feature patterns for determining the clinical outcome of a disease such as, for example, the probability of cure, sustenance, relapse, worsening, and/or mortality for COVID-19.

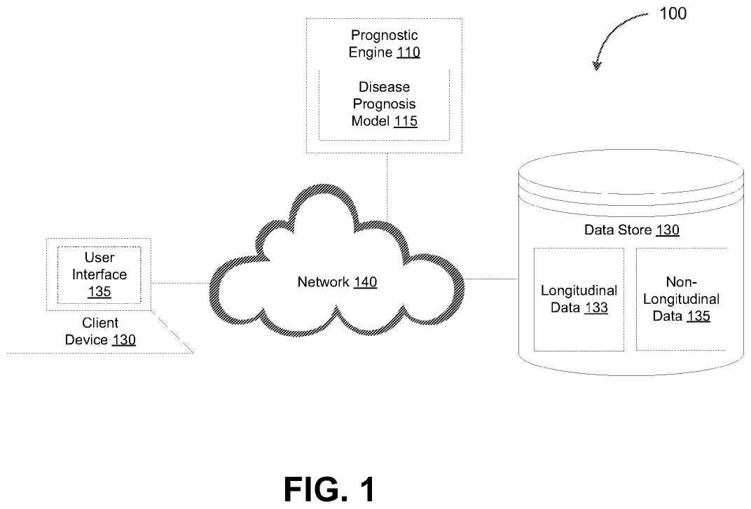

FIG. 1 depicts a system diagram illustrating an example of a prognosis system 100, in accordance with some example embodiments. Referring to FIG. 1, the prognosis system 100 may include a prognostic engine 110, a client device 120, and a data store 130. As shown in FIG. 1, the prognostic engine 110, the client device 120, and the data store 130 may be communicatively coupled via a network 140. The client device 120 may be a processor-based device including, for example, a workstation, a desktop computer, a laptop computer, a smartphone, a tablet computer, a wearable apparatus, and/or the like. The data store 130 may be a relational database, a non-structured query language (NoSQL) database, an in-memory database, a graph database, a key-value store, a document store, and/or the like. The network 140 may be a wired network and/or a wireless network including, for example, a local area network (LAN), a virtual local area network (VLAN), a wide area network (WAN), a public land mobile network (PLMN), the Internet, and/or the like.

In some example embodiments, the prognostic engine 110 may apply, to a longitudinal data 133 from the data store 130, a disease prognosis model 115 in order to determine a clinical outcome of a disease for a patient associated with the longitudinal data 133. For example, in some cases, the longitudinal data 133 may include a sequence {xᵢ|x₁, x₂, . . . , x_{T}}, wherein T denotes the length of the sequence (or the quantity of timepoints) and xᵢ denotes the data at the i-th timepoint. In some cases, xᵢ may be a health record (e.g., an electronic health record (EHR)) represented as a vector, a matrix, or a tensor. Accordingly, the longitudinal data 133 may include, for example, a first health record x₁ of the patient from a first timepoint t₁ and a second health record x₂ of the patient from a second timepoint t₂. The disease prognosis model 115 may be trained to determine, based on the sequence {xᵢ|x₁, x₂, . . . , x_{T}} included in the longitudinal data 133, a clinical outcome for the disease that includes, for example, a probability of cure, sustenance, relapse, worsening, and/or mortality for the patient associated with the longitudinal data 133. In some cases, the clinical outcome prediction performed by the disease prognosis model 115 may be formulated as a sequence-to-one problem. For instance, the disease prognosis model 115 may extract, from the longitudinal data 133, a feature set (e.g., hidden features) representative of the short-term dependencies present within the longitudinal data 133. Furthermore, the disease prognosis model 115 may determine the importance of each feature in the feature set for each timepoint in the longitudinal data 133. The clinical outcome for the disease may be determined based on the importance of each feature in the feature set for each timepoint in the longitudinal data 133.

The aforementioned sequence-to-one problem for disease outcome prediction may be challenging due to the presence of various features (e.g., disease symptoms) at different timepoints. In some instances, the features (e.g., disease symptoms) that are present at earlier timepoints may be correlated with those that are present at later timepoints. While an accurate prognosis requires a holistic analysis of feature-wise information as well as temporal information, existing approaches to outcome prediction are able to account for either time-dependent feature importance or spatial feature importance but not both. By contrast, various implementations of the disease prognosis model 115 disclosed herein includes a spacetime attention 200 trained to jointly weigh feature importance over the time axis and the feature space.

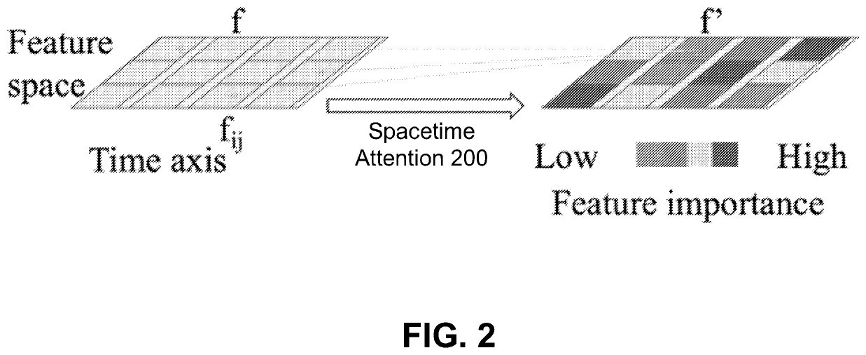

FIG. 2 depicts a schematic diagram illustrating an example of the spacetime attention 200 calculating feature importance across a time dimension as well as a feature dimension, in accordance with some example embodiments. As shown in FIG. 2, the feature importance of a feature f at any particular timepoint may be calculated based on each feature's value at every timepoint fᵢⱼ. In some cases, the spacetime attention 200 may be implemented as one or more two-dimensional convolutional layers trained to computes keys, queries, and values for each feature extracted from the longitudinal data 133 using convolutional filters (e.g., 1×1 convolutional filters) to calculate the alignment score as feature importance. The benefit of using 1×1 convolutional filters for attention calculation is the joint weighting of feature importance from two dimensions including the time dimension and the feature dimension. Equation (1) below shows that the features extracted from the longitudinal data 133 (e.g., hidden features) may be adjusted by the spacetime attention 200 with a weighting factor γ.

$${f}^{′} = f + γ \cdot a ( f ) \qquad ( 1 )$$

wherein f denotes the hidden features extracted from the longitudinal data 133 (in a manner described in more detail below), f′ denotes each features adjusted value as determined by the spacetime attention a(·).

Equation (2) below shows the calculation of the spacetime attention a(·).

$$a ( f ) = {\sum }_{i = 1}^{H} {\sum }_{j = 1}^{T} softmax ( q ( {f}_{i j} ) \cdot k ( f ) ) \cdot v ( f ) \qquad ( 2 )$$

wherein H denotes the size of the feature space extracted from the longitudinal data, T denotes the quantity of timepoints in the longitudinal data 133. As shown in Equation (2), the feature importance of a given feature f is determined based on other features {fᵢⱼ|i=1,2, . . . , H; j=1,2, . . . , T}. In some cases, feature importance may be measured by calculating an alignment score via the key k( ), value v( ), and query q( ) operations implemented by the convolution filters (e.g., 1×1 convolution filters).

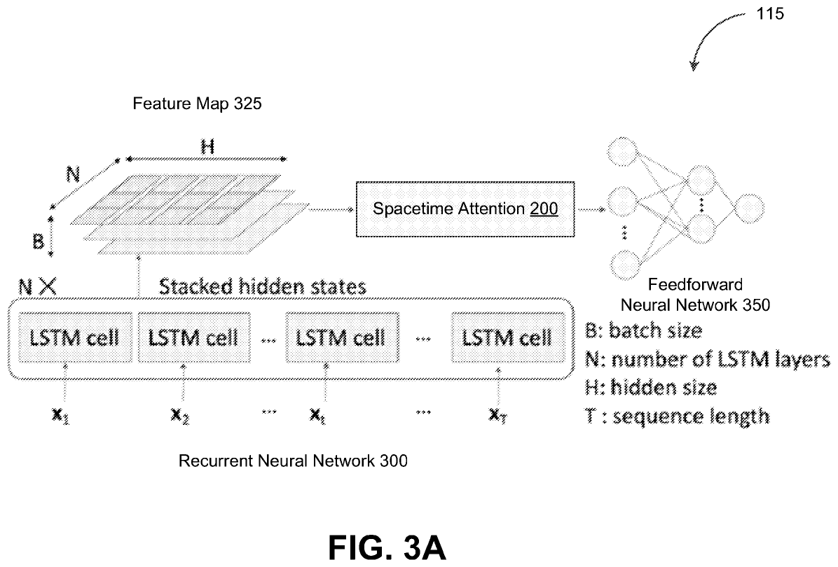

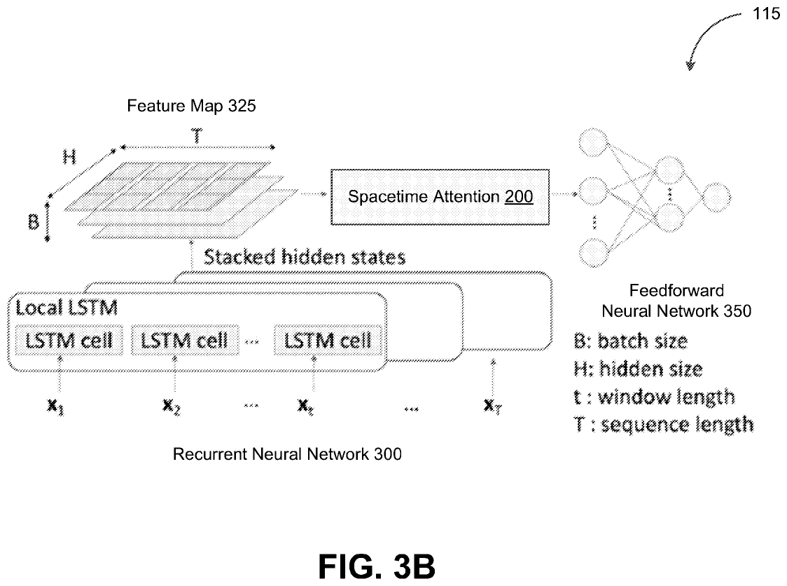

As noted, the disease prognosis model 115 may extract, from the longitudinal data 133, a feature set (e.g., hidden features) representative of the short-term dependencies present within the longitudinal data 133. In some cases, the disease prognosis model 115 may include a recurrent neural network (RNN) trained to extract the feature set from the longitudinal data 133 before the spacetime attention 200 is applied to determine the importance of each feature for every timepoint in the longitudinal data 133. FIGS. 3A-B depict schematic diagram illustrating examples of the disease prognosis model 115 in which the spacetime attention 200 is integrated into a recurrent neural network 300. In some cases, the recurrent neural network 300 may be a bi-directional recurrent neural network (RNN), a long short-term memory (LSTM) network, a local-long short-term memory (LSTM) network with a given window size for timepoints, a gated recurrent unit (GRU) network, and/or the like.

Referring to FIG. 3A, in some cases, the recurrent neural network 310 may be a long short-term memory (LSTM) network trained to extract, from the longitudinal data 133, short-term and long-term dependencies to form a feature map 325. The size of the feature map 325 may be N×H, where N corresponds to the quantity of stacked recurrent layers and H corresponds to quantity of features in the hidden state of the long-short term memory (LSTM) network. The spacetime attention 200 may operate on the feature map 325 to learn the correlation between the H quantity of features across the T quantity of timepoints. As shown in FIG. 3A, the spacetime attention 200 may output, for ingestion by a feedforward neural network 350, an adjusted feature map indicating the importance of each of the H quantity of features at each of the T quantity of timepoints. For example, in some cases, the adjusted feature map may indicate a first importance of a first feature at the first timepoint and a second importance of the first feature at the second timepoint. Furthermore, in some cases, the adjusted feature map may indicate a third importance of a second feature at the first timepoint and a fourth importance of the second feature at the second timepoint. The feedforward neural network 350, which in some cases may be a multi-layer perceptron model, may operate on the adjusted feature map to determine the clinical outcome for the disease.

While the spacetime attention 200 may be proficient at learning long-term dependencies, it may lack the capability of modeling short-term (or local) dependencies in order. Some attention-based models, such as transformers, rely on positional embeddings to encode the order of short-term dependencies. However, unlike an image or a sentence where the sequential ordering of elements has contextual meanings, the health records (e.g., electronic health records (EHR)) included in the longitudinal data 133 are not strictly ordered. For example, a patient could undergo the lab tests before undergoing medical imaging examination or vice versa. As such, positional embeddings are not suitable for encoding the order of the short-term dependencies present in the longitudinal data 133. Nevertheless, long short-term memory (LSTM) networks do not impose strict ordering at least because the signals stored in the memory cells can still propagate when local order undergoes change. To disentangle the learning of short-term and long-term dependencies, the learning of short-term dependencies may be restricted to a set of local long short term memory (LSTM) networks and the learning of long-term dependencies may be restricted to the spacetime attention 200. An example of the disease prognosis model 115 in which the recurrent neural network 115 is implemented as a set of local long short term memory (LSTM) networks is shown in FIG. 3B. The local long short term memory (LSTM) networks are limited to learning the sequential patterns that are present within a particular window size and extracting the corresponding local patterns as hidden features.

Referring again to FIG. 3B, after concatenating the hidden states from each local long short term memory (LSTM) network, the stacked hidden states may become a matrix containing a T quantity of vectors of length H. The spacetime attention 200 then refines the hidden states by mining the feature-wise and long-term dependencies before outputting a set of adjusted hidden states for ingestion by the feedforward neural network 350 (e.g., the multi-layer perceptron model) for a determination of clinical outcome. In some cases, the set of local long short term memory (LSTM) networks and the spacetime attention 200 may form an R-Transformer, which differs from the transformer models used in computer vision and natural language processing where location information are encoded with the positional embeddings.

In some example embodiments, the prognostic engine 110 may apply the disease prognosis model 115 to a combination of the longitudinal data 133 and non-longitudinal data in order to determine the clinical outcome of the disease for the patient associated with the longitudinal data 133 and the non-longitudinal data 135. In this context, the non-longitudinal data 135 may include data whose values remain fixed across different timepoints. Examples of the non-longitudinal data 135 may include demographic information, medical history (e.g., preexisting conditions such as hypertension, obesity, hyperlipidemia, diabetes mellitus, and/or the like), medical image data (e.g., a grading of disease severity as portrayed in one or more medical images), and/or electrogram data (e.g., a grading of disease severity as indicated by one or more electrograms). The prognosis engine 110 may combine the longitudinal data 133 with the non-longitudinal data 135 by at least concatenating the non-longitudinal data 135 with the longitudinal data 133 from each timepoint. For example, the non-longitudinal data 135 associated with the patient may be concatenated with the first health record x₁ from the first timepoint t₁ and the second health record x₂ from the second timepoint t₂.

In some example embodiments, the prognostic engine 110 may preprocess the longitudinal data 133 and/or the non-longitudinal data 135 prior to applying the disease prognosis model 115. For example, the preprocessing of the longitudinal data 133 and/or the non-longitudinal data 135 may include normalizing one or more of the values present therein. For at least some medical images, the preprocessing performed by the prognostic engine 110 may include determining a metric quantifying a severity of the disease portrayed in the medical images. In the case of chest X-rays, for example, the prognostic engine 110 may compute a radiographic assessment of lung oedema (RALE) score to characterize the disease severity of acute respiratory distress syndrome (ARDS) in patients positive for COVID-19. For numeric variables, the prognostic engine 110 may apply min-max normalization to render the values of each variable on the same scale (e.g., of zero to one). The values of binary variables present in the longitudinal data 133 and/or the non-longitudinal data 135 may be presented as one of two values (e.g., 0 and 1).

In some cases, the longitudinal data 133 and the non-longitudinal data 135 may exhibit sparsity where the values of one or more variables are missing. Accordingly, in some example embodiments, the preprocessing of the longitudinal data 133 and/or the non-longitudinal data 135 may include excluding, from further analysis, one or more variables that are available for less than a threshold quantity of patients (e.g., 95% of the patients). Moreover, in instances where the prognostic engine 110 detects that the non-longitudinal data 135 of the patient includes one or more missing values for a non-longitudinal variable (e.g., a variable having a static value across timepoints), the prognostic engine 110 may perform mean-filling in which the one or more missing values are replaced with a mean value of the variable observed in the available dataset (e.g., the mean value of the variable associated with other patients). Where the prognostic engine 110 detects that the longitudinal data 133 of the patient includes a missing value for a longitudinal variable (e.g., a variable having a fluctuating value across timepoints) at a first timepoint, the prognostic engine 110 may perform a forward filling in which the missing value is replaced with a first value of the longitudinal variable from a second timepoint preceding the first timepoint. Alternatively, the prognostic engine 110 may perform a backward filling in which the missing value is replaced with a second value of the longitudinal variable from a third timepoint following the first timepoint. In some cases, the missing value for the longitudinal variable at the first timepoint may be interpolated based on the first value of the longitudinal variable from the second timepoint preceding the first timepoint and the second value of the longitudinal variable from the third timepoint following the first timepoint.

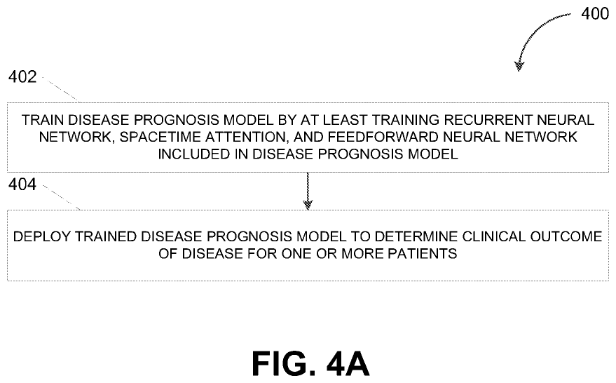

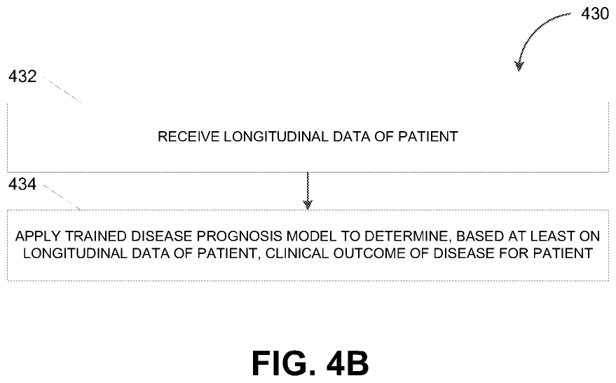

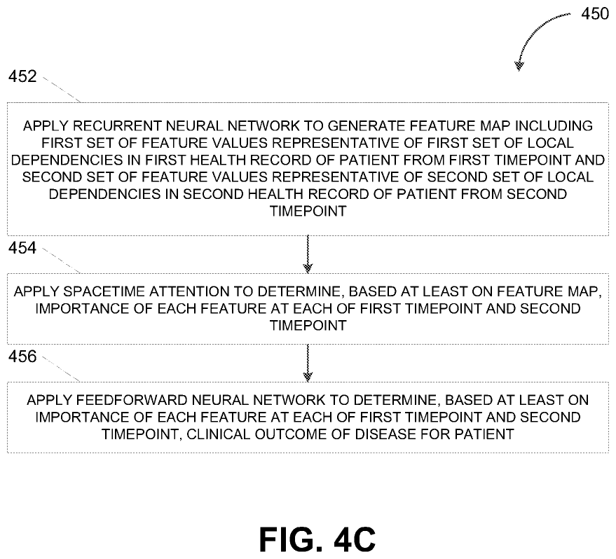

FIG. 4A depicts a flowchart illustrating an example of a process 400 for training the disease prognosis model 115 to perform clinical outcome prediction, in accordance with some example embodiments. Referring to FIGS. 1-2, 3A-B, and 4A, the process 400 may be performed, for example, by the prognostic engine 110 to train the disease prognosis model 115.

At 402, the prognostic engine 110 may train the disease prognosis model 115 by at least training the recurrent neural network 300, the spacetime attention 200, and the feedforward neural network 350 included in the disease prognosis model 115. For example, in some example embodiments, the disease prognosis model 115 may be trained to determine, based at least on the longitudinal data 133 of the patient, a clinical outcome of a disease for the patient. As shown in FIGS. 3A-B, the disease prognosis model 115 may include the recurrent neural network 330, the spacetime attention 200, and the feedforward neural network 350. Moreover, the longitudinal data 133 may include, for each timepoint in a sequence of timepoints, a corresponding health record. Accordingly, the training of the disease prognosis model 115 may include training the recurrent neural network 330, the spacetime attention 200, and the feedforward neural network 350. For instance, the recurrent neural network 330 may be trained to extract, from each health record included in the longitudinal data 133, a feature set representative of one or more local dependencies present within the health record. The spacetime attention 200 may be trained to determine an importance of each feature in the feature set at each timepoint in the sequence of timepoints. The feedforward neural network 350 may be trained to determine, based at least on the importance of each feature in the feature set at each time point in the sequence of timepoints, the clinical outcome of the disease.

At 404, the prognosis engine 110 may apply the trained disease prognosis model 115 to determine a clinical outcome of a disease for one or more patients. In some example embodiments, the trained disease prognosis model 115 may be applied to determine the clinical outcome of a variety of diseases including, for example, coronavirus disease (COVID-19), Alzheimer's disease, age-related macular degeneration, and/or the like. The output of the trained disease prognosis model 115 may include, as the clinical outcome for the disease, a probability associated with one or more of cure, sustenance, relapse, worsening, and mortality.

FIG. 4B depicts a flowchart illustrating an example of a process 430 for machine learning enabled clinical outcome prediction, in accordance with some example embodiments. Referring to FIGS. 1-2, 3A-B, and 4A-B, the process 430 may be performed by the prognostic engine 110 and may implement operation 404 of the process 400 shown in FIG. 4A.

At 432, the prognosis engine 110 may receive the longitudinal data 133 of the patient. For example, in some example embodiments, the prognosis engine 110 may receive, as a part of the longitudinal data 133 of the patient, a first health record of the patient at a first timepoint and a second health record of the patient at a second point. In some cases, the longitudinal data 133 of the patient may be combined with the non-longitudinal data 135 of the patient. The non-longitudinal data 135 of the patient may include one or more non-longitudinal variables whose values remain static over successive timepoints whereas the longitudinal data 133 of the patient may include one or more longitudinal variables whose values fluctuate over the successive timepoints. The combining of the longitudinal data 133 and the non-longitudinal data 135 of the patient may therefore include concatenating the values of the longitudinal variables at each timepoint (e.g., included in the health record associated with each timepoint) with the values of the non-longitudinal variables.

At 434, the prognosis engine 110 may apply the trained disease prognosis model 115 to determine, based at least on the longitudinal data 133 of the patient, a clinical outcome of a disease for the patient. For example, in some example embodiments, the trained disease prognosis model 115 may be applied to determine, based at least on the first health record of the patient at the first timepoint and the second health record of the patient at the second timepoint, a clinical outcome of a disease for the patient. The disease prognosis model 115, which includes the spacetime attention 200 integrated into the recurrent neural network 300, may be capable of identifying the short-term as well as long-term dependencies present within the longitudinal data 133. In particular, the recurrent neural network 300 may be trained to extract a variety of features representative of the short-term dependencies present within the longitudinal data 133 while the spacetime attention 200 may be trained to recognize the changes in the feature importance of each feature across different timepoints. Doing so may enable the disease prognosis model 115 to generate an accurate prognosis of the clinical outcome of the disease for the patient.

FIG. 4C depicts a flowchart illustrating an example of a process 450 for machine learning enabled clinical outcome prediction, in accordance with some example embodiments. Referring to FIGS. 1-2, 3A-B, and 4A-C, the process 450 may be performed by the disease prognosis model 115 and may implement operation 434 of the operation 430 shown in FIG. 4B.

At 452, the disease prognosis model 115 may apply the recurrent neural network 300 to generate a feature map including a first set of feature values for a hidden feature set representative of a first set of local dependencies present within a first health record of the patient from a first timepoint and a second set of feature values the hidden feature set representative of a second set of local dependencies present within a second health record of the patient from a second timepoint. In some example embodiments, the recurrent neural network 300 may be trained to extract local (or short-term) dependencies present within, for example, the first health record x₁ from the first timepoint t₁ and the second health record x₂ from the second timepoint t₂ in the sequence {xᵢ|x₁, x₂, . . . , x_{T}} included in the longitudinal data 133. As shown in FIGS. 3A-B, the recurrent neural network 300 may extract, from each of the first health record x₁ and the second health record x₂, an H quantity of features (e.g., hidden features). Accordingly, FIGS. 3A-B further show that the output of the recurrent neural network 300 may be the feature map 325 that is N×H in size. For example, the feature map 325 may include, for the health record from each of the T quantity of timepoints, a corresponding vector of length H. In cases where the recurrent neural network 300 is implemented as a local long short term memory (LSTM) network, the recurrent neural network 300 may be limited to learning the sequential patterns that are present within a particular window size and extracting the corresponding local patterns as hidden features.

At 454, the disease prognosis model 115 may apply the spacetime attention 200 to determine, based at least on the feature map, the importance of each feature at each of the first timepoint and the second timepoint. In some example embodiments, the spacetime attention 200 may operate on the feature map 325 to determine the correlation between the H quantity of features across the T quantity of timepoints and output a corresponding adjusted feature map indicating the importance of each of the H quantity of features at each of the T quantity of timepoints. For example, the spacetime attention 200 may determine the first importance of a first feature at the first timepoint t₁ as well as the second importance of the first feature at the second timepoint t₂. Moreover, the spacetime attention 200 may determine the third importance of a second feature at the first timepoint t₁ and the fourth importance of the second feature at the second timepoint t₂.

As noted, that the importance of different features may vary over time is consistent with clinical observations such as in COVID-19 patients where the fibrotic-like abnormalities that are common during the first three months will have mostly disappeared by the one year mark while consolidations are usually resolved within six months. Accordingly, the adjusted feature map may include adjusted feature values for each of the first feature and the second feature. For instance, the values of the first feature at the first timepoint t₁ and the second timepoint t₂ may be adjusted based on the first importance of the first feature at the first timepoint t₁ and the second importance of the first feature at the second timepoint t₂, respectively. The values of the second feature at the first timepoint t₁ and the second timepoint t₂ may likewise be adjusted based on the third importance of the second feature at the first timepoint t₁ and the fourth importance of the second feature at the second timepoint t₂, respectively.

At 456, the disease prognosis model 115 may apply the feedforward neural network 350 to determine, based at least on the importance of each feature at each of the first timepoint and the second timepoint, a clinical outcome of a disease for the patient. In some example embodiments, the disease prognosis model 115 may apply the feedforward neural network 350, which may operate on the adjusted feature map to determine the clinical outcome of the disease for the patient. For example, the feedforward neural network 350 may be implemented as a multi-layer perceptron model. Moreover, the feedforward neural network 350 operating on the adjusted feature map may determine, based at least on the importance of each feature at different timepoints in the longitudinal data 133, the clinical outcome of the disease for the patient.

In some example embodiments, the performance of the disease prognosis model 115 in predicting the clinical outcome of COVID-19 was evaluated based on data associated with a cohort of 365 patients hospitalized with severe COVID-19 pneumonia. The non-longitudinal data 135 for each patient, which are collected upon initial admission, includes demographic information, medical history, and medical image data (e.g., radiographic assessment of lung oedema (RALE) quantifying disease severity reflected by chest X-rays). The longitudinal data 133 associated with each patient, which includes laboratory test results and vital signs, are collected during follow-up visits. The average quantity of timepoints for each patient is 10 with a standard deviation of 6. The clinical outcome for each patient, such as survival status, are collected on the 60th day after initial hospitalization. Table 1 below shows the patient characteristics upon initial admission to hospital.

TABLE 1

| Characteristics | Value |
|---|---|
| Total number of patients | 365 |
| Deceased after 60 days | 65 |
| Mean age, years | 58 ± 13 |

Sex

| | |
|---|---|
| Male | 217 |
| Female | 149 |
| Mean body mass index, kg/m² | 28 ± 13 |
| Medical history | |
| Diabetes mellitus | 69 |
| Hyperlipidemia | 70 |
| Obesity | 113 |
| Hypertension | 192 |
| Radiographic assessment of lung oedema (RALE) | 15 ± 13 |
| Vital signs* | |
| Systolic Blood Pressure, millimeters of | |
| mercury | 124 ± 17 |
| Diastolic Blood Pressure, millimeters of | |
| mercury | 72 ± 10 |
| Pulse Rate, beats per minute | 76 ± 16 |
| Respiratory Rate, breaths per minute | 23 ± 6 |
| Laboratory test* | |

Table 2 below shows the laboratory test variables from an example patient on day one. As shown in Table 2, laboratory test variables may include Fibrinogen, C Reactive Protein, Pro-thrombin International Normalized Ratio, Prothrombin Time, Lactate Dehydrogenase, D-Dimer, Albumin, Ferritin, Alanine Aminotransferase, Aspartate Aminotransferase, Chloride, Protein, Alkaline Phosphatase, Bilirubin, Calcium, Creatinine, Glucose, Hematocrit, Hemoglobin, Potassium, Platelets, Erythrocytes, Sodium, and Leukocytes.

TABLE 2

| Laboratory test variable name | Value | Units |
|---|---|---|
| Albumin | 3.2 | g/dL |
| Alkaline Phosphatase | 80 | U/L |
| Alanine Aminotransferase | 26 | U/L |
| Activated Partial Thromboplastin Time | 36 | sec |
| Aspartate Aminotransferase | 21 | U/L |
| Basophils | 0.1 | 10\hat{ }3/uL |
| Bilirubin | 0.7 | mg/dL |
| N-Terminal ProB-type Natriuretic Peptide | 171.9 | pg/mL |
| Blood Urea Nitrogen | 15 | mg/dL |
| Calcium | 8.5 | mg/dL |
| Chloride | 100 | mEq/L |
| Carbon Dioxide | 28 | mEq/L |
| Creatinine | 0.6 | mg/dL |
| C Reactive Protein | 86.1 | mg/L |
| D-Dimer | 0.35 | ug/mL FEU |
| Eosinophils | 0 | 10\hat{ }3/uL |
| Ferritin | 1815 | ng/mL |
| Fibrinogen | 662 | mg/dL |
| Glucose | 66 | mg/dL |
| Hematocrit | 40.1 | % |
| Hemoglobin | 13.9 | g/dL |
| Prothrombin International Normalized Ratio | 1.3 | RATIO |
| Potassium | 3.6 | mEq/L |
| Lactate Dehydrogenase | 229 | U/L |
| Lymphocytes | 1.2 | 10\hat{ }3/uL |
| Monocytes | 0.6 | 10\hat{ }3/uL |
| Neutrophils | 6.5 | 10\hat{ }3/uL |
| Phosphate | 2.3 | mg/dL |
| Platelets | 314 | 10\hat{ }3/uL |
| Protein | 6.4 | g/dL |
| Prothrombin Time | 16 | sec |
| Erythrocytes | 4.43 | 10\hat{ }6/uL |
| Soluble Interleukin 1 Receptor-Like 1 | 6770 0 | pg/mL |
| Sodium | 139 | mEq/L |
| Troponin T | 0.01 | ng/mL |
| Unspecified Cells | 0 | 10\hat{ }3/uL |
| Urate | 1.9 | mg/dL |
| Leukocytes | 8.3 | 10″3/uL |

In some example embodiments, the performance of the disease prognosis model 115 may be evaluated by training, validating, and testing the disease prognosis model 115 using a training set, a validation set, and a testing set generated by splitting the aforementioned dataset with a ratio of 7:1:2. In some cases, the training of the disease prognosis model 115 may include stochastic gradient optimization while the validation of the disease prognosis model 115 may include tuning the hyperparameters of the disease prognosis model 115 on the validation set. For example, in cases where the disease prognosis model 115 includes an R-Transformer formed by a set of local long short term memory (LSTM) networks and the spacetime attention 200, a window size of 6 may be used to limit the local long short term memory (LSTM) networks to learning short-term dependencies. Furthermore, the size of the hidden feature set (e.g., the value of H) may be set, in some cases, to 32. With the tuned hyperparameters, the disease prognosis model 115 is subjected to 50 epochs of training with a batch size of 2. The training of the disease prognosis model 115 may also be subjected to a learning rate schedule, such as an annealed learning rate that gradually decreased from 1e−3 to 1e−5. The disease prognosis model 115 with the best performance on the validation set is evaluated on the testing set.

In some example embodiments, the evaluation of the performance of the disease prognosis model 115 may include assessing the prognostic values of different data modalities by incrementally incorporating different data modalities into disease prognosis model 115. For example, the spacetime attention 200 for clinical outcome prediction may be interpreted by first visualizing the spacetime feature map (e.g., the varying importance of different features across successive timepoints). The critical timepoints identified by the spacetime attention 200 (e.g., the timepoints with the highest feature importance) may be compared against those identified by the Acute Physiology and Chronic Health Evaluation II (APACHE II) system (e.g., timepoints with the highest increase of Apache II score). The latter is a clinical nomogram to quantify disease severity. By measuring the physiologic variables, age and previous health status, Apache II gives a score between 0 to 71 where higher scores indicate a higher risk of death. A Mann-Whitney U test is performed to compare the critical timepoints identified by the spacetime attention 200 and by the Apache II system.

Table 3 shows the area under the curve (AUC) when the disease prognosis model 115 (e.g., implemented using a long short term memory (LSTM) network) is used to determine the clinical outcome of COVID-19 patients. As shown in Table 3, when the disease prognosis model 115 operates on laboratory test data alone (as one type of the longitudinal data 133), the disease prognosis model 115 is able to achieve an area under the curve (AUC) of 0.63 on the testing set. With the incorporation of vital signs (as another type of the longitudinal data 133), the performance of the disease prognosis model 115 increased to an area under the curve (AUC) of 0.70. These two results demonstrate the effectiveness of the disease prognosis model 115 in modeling longitudinal data. Moreover, the incorporation of different types of the non-longitudinal data 135 (e.g., static data including demographic data, medical history data, and medical data), further improved the performance of the disease prognosis model 115 to an area under the curve (AUC) of 0.73, 0.75, and 0.76, respectively.

TABLE 3

| Input modalities | | | | | |
|---|---|---|---|---|---|
| | | | Medical | | |
| Lab test | Vital signs | Demographic | history | RALE | AUC |
| X | | | | | 0.63 |
| X | X | | | | 0.70 |
| X | X | X | | | 0.73 |
| X | X | X | X | | 0.75 |
| X | X | X | X | X | 0.76 |

In some example embodiments, the performance of the disease prognosis model 115 is further evaluated for the different network architectures shown in FIGS. 3A-B (e.g., long short term memory (LSTM) network with the spacetime attention 200 and R-Transformer with the spacetime attention 200). The performance of the disease prognosis model 115 is also compared against that of two conventional models including a long short term memory (LSTM) network with embedded temporal attention and a transformer with embedded spatial attention. Table 4 shows the performance of the different models.

TABLE 4

| Model name | AUC |
|---|---|
| Clinical model | 0.61 |
| LSTM | 0.76 |
| LSTM with temporal attention | 0.77 |
| LSTM with spacetime attention | 0.80 |
| R-Transformer with spacetime attention | 0.94 |

The clinical model in Table 4, which considers only the non-longitudinal data collected at initial admission to hospital achieves an area under the curve (AUC) of 0.61. The poor performance of the clinical model may be attributable to its inability to account for nonlinear interactions among variables and exclusion of longitudinal data. A conventional long short term memory (LSTM) network alone is able to achieve an area under the curve (AUC) of 0.76 and an area under the curve (AUC) of 77 with the assistance of temporal attention. With the addition of the spacetime attention 200, the disease prognosis model 115 has an area under the curve of 0.80 when implemented using a long short term memory (LSTM) network and an area under the curve of 0.94 when implemented as an R-transformer. These result indicate that separating the learning of short-term dependencies and long-term dependencies, as is the case when the disease prognosis model 115 is implemented using an R-transformer, can enhance the accuracy of clinical outcome prediction.

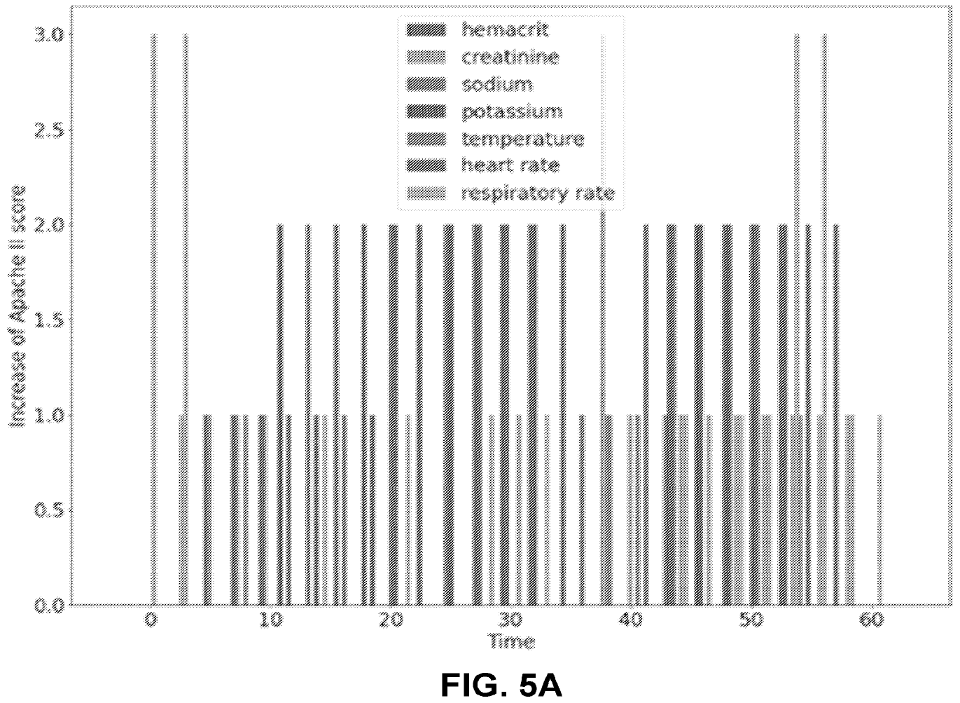

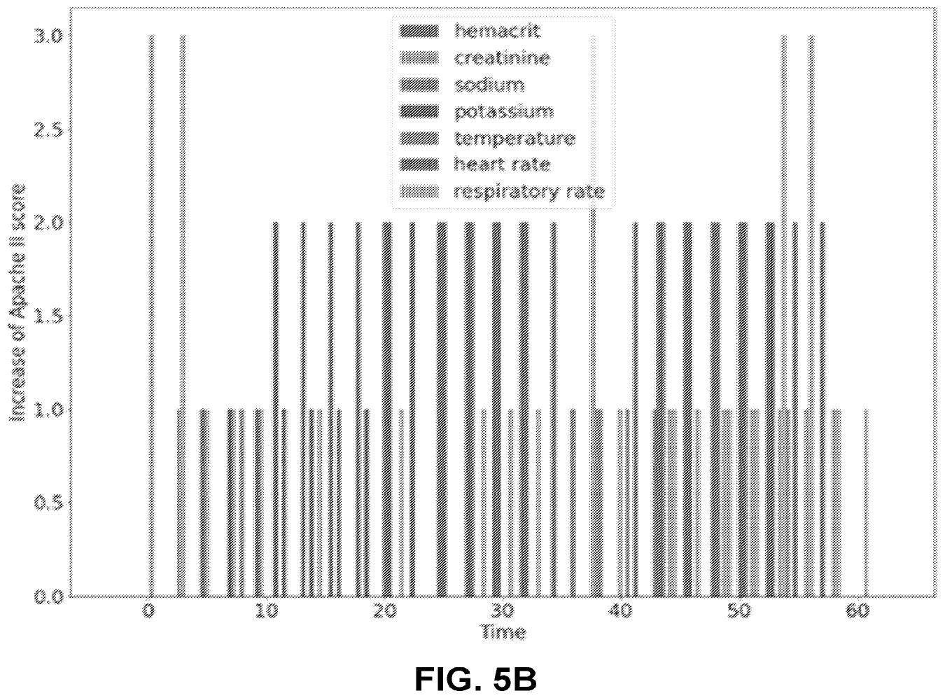

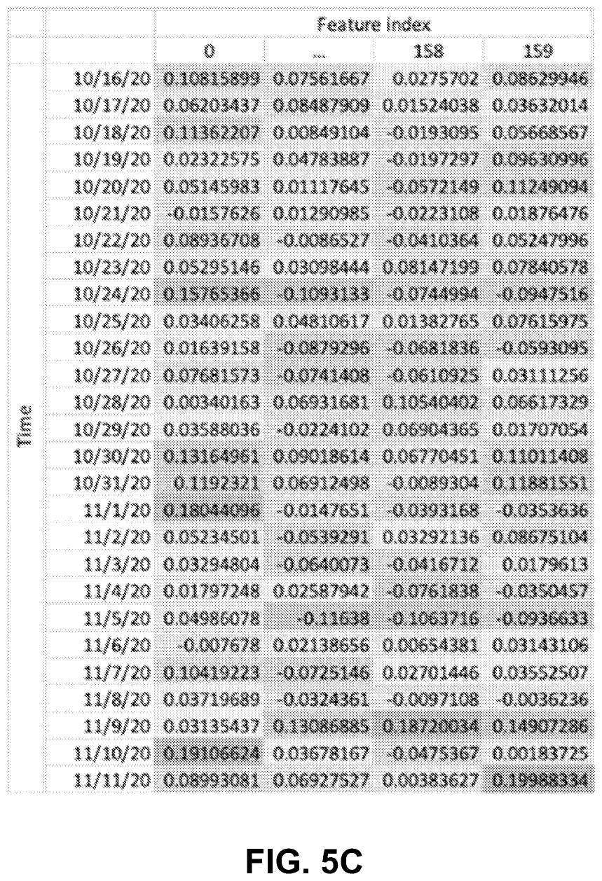

The adjusted feature map output by the spacetime attention 200 may include a variety of nonlinear interactions between features. Explaining the spacetime attention 200 with the hidden features may be less straightforward and intuitive than explaining variables with physical meanings, such as heart rate, body temperature, and/or the like. Thus, in some example embodiments, the Apache II system may be used as a bridge to interpret where the spacetime attention 200 is looking at. FIG. 5A depicts a graph illustrating an example of one patient's Apache II score over time. FIG. 5B depicts a graph illustrating the increase of Apache II score decomposed by different physiologic variables for that patient. FIG. 5C depicts a table providing a visualization of an example output of the spacetime attention 200. As show in FIG. 5C, the importance of each individual feature may vary not only across successive timepoints but also from that of other features.

Based on the statistical testing of critical time points identified by the spacetime attention 200 and the Apache II system, the values of the features present at the initial timepoint (e.g., corresponding to disease onset) and the final timepoint (e.g., corresponding to the most recent condition) tend to have more importance than those from other timepoints in determining the clinical outcome of COVID-19 for a patient, including the likelihood of persistent and/or recurring symptoms. The significance of the initial time points is consistent with the finding that experiencing more than five symptoms during the first week of illness was associated with COVID-19. Significantly (p<0.05) correlated critical time points for the clinical outcome of COVID-19 between the spacetime attention 200 and the Apache II system include i) when the respiratory rate is abnormal at the initial timepoint and the 60-day timepoint, and ii) when the heart rate and creatinine level are both in abnormal ranges at any timepoint.

In view of the above-described implementations of subject matter this application discloses the following list of examples, wherein one feature of an example in isolation or more than one feature of said example taken in combination and, optionally, in combination with one or more features of one or more further examples are further examples also falling within the disclosure of this application:

Item 1: A computer-implemented method, comprising: training a disease prognosis model to determine, based at least on longitudinal data, a clinical outcome of a disease, the longitudinal data including a health record for each timepoint in a sequence of timepoints, the training of the disease prognosis model includes training a recurrent neural network, a spacetime attention, and a feedforward neural network, the recurrent neural network being trained to extract, from each health record, a feature set representative of one or more local dependencies present within the health record, the spacetime attention being trained to determine an importance of each feature in the feature set at each timepoint in the sequence of timepoints, and the feedforward neural network being trained to determine, based at least on the importance of each feature in the feature set at each time point in the sequence of timepoints, the clinical outcome of the disease; and applying the trained disease prognosis model to determine, based at least on a first health record from a first timepoint and a second health record from a second timepoint, the clinical outcome of the disease for a patient associated with the first health record and the second health record.

Item 2: The method of Item 1, wherein the recurrent neural network is a bi-directional recurrent neural network (RNN), a long short-term memory (LSTM) network, a local long short-term memory (LSTM) network with a given window size for timepoints, or a gated recursive unit (GRU) network.

Item 3: The method of any of Items 1 to 2, wherein the feedforward neural network is a multi-layer perceptron model.

Item 4: The method of any of Items 1 to 3, wherein the trained disease prognosis model determines the clinical outcome of the disease for the patient associated with the first health record and the second health record by at least applying the trained recurrent neural network to extract, from the first health record, a first set of feature values for a hidden feature set representative of a first set of local dependencies present within the first health record, and applying the trained recurrent neural network to extract, from the second health record, a second set of feature values for the hidden feature set representative of a second set of local dependencies present within the second health record.

Item 5: The method of Item 4, the trained recurrent neural network outputs, for ingestion by the trained spacetime attention, a feature map comprising the first set of feature values from the first timepoint and the second set of feature values from the second timepoint.

Item 6: The method of any of Items 4 to 5, wherein the trained disease prognosis model further determines the clinical outcome of the disease for the patient associated with the first health record and the second health record by at least applying the trained spacetime attention to determine, based at least on a feature map including the first set of feature values and the second set of feature values, the importance of each feature in the hidden feature set at each of the first timepoint and the second timepoint.

Item 7: The method of Item 6, wherein the trained spacetime attention includes one or more two-dimensional convolutional layers trained to determine the importance of each feature in the hidden feature set across a time dimension and a feature dimension.

Item 8: The method of Item 7, wherein the one or more two-dimensional convolutional layers include 1×1 convolutional filters configured to perform a joint weighting of the importance of each feature in the hidden feature set across the time dimension and the feature dimension.

Item 9: The method of any of Items 6 to 8, wherein the trained spacetime attention determines, for a first feature from the hidden feature set, a first importance of the first feature at the first timepoint and a second importance of the first feature at the second timepoint.

Item 10: The method of Item 9, wherein the trained spacetime attention further determines, for a second feature from the hidden feature set, a third importance of the second feature at the first timepoint and a fourth importance of the second feature at the second timepoint.

Item 11: The method of any of Items 6 to 10, wherein the trained disease prognosis model further determines the clinical outcome of the disease for the patient associated with the first health record and the second health record by at least applying the trained feedforward neural network to determine, based at least on the importance of each feature in the hidden feature set at each of the first timepoint and the second timepoint, the clinical outcome of the disease for the patient.

Item 12: The method of any of Items 1 to 11, wherein the disease prognosis model is further trained to determine the clinical outcome of the disease based on non-longitudinal data, wherein the non-longitudinal data is static across the sequence of timepoints, and wherein the non-longitudinal data is concatenated with the health record associated with each timepoint in the sequence of timepoints.

Item 13: The method of Item 12, further comprising: identifying one or more missing values for a non-longitudinal variable comprising the non-longitudinal data; and replacing the one or more missing values with a mean value of the non-longitudinal variable observed in an available dataset.

Item 14: The method of any of Items 12 to 13, wherein the non-longitudinal data includes at least one of demographic information and medical history.

Item 15: The method of any of Items 12 to 14, wherein the non-longitudinal data includes medical image data and/or electrogram data corresponding to a metric quantifying a severity of the disease portrayed in one or more medical images and/or electrograms.

Item 16: The method of any of Items 1 to 15, wherein the health record associated with each timepoint in the sequence of timepoints includes a value for each of a plurality of vital sign statistics.

Item 17: The method of Item 16, wherein the plurality of vital sign statistics include one or more of systolic blood pressure, diastolic blood pressure, pulse rate, and respiratory rate.

Item 18: The method of any of Items 1 to 17, wherein the health record associated with each timepoint in the sequence of timepoints includes a value for each of a plurality of laboratory test variables.

Item 19: The method of Item 18, wherein the plurality of laboratory variables include one or more of Fibrinogen, C Reactive Protein, Pro-thrombin International Normalized Ratio, Prothrombin Time, Lactate Dehydrogenase, D-Dimer, Albumin, Ferritin, Alanine Aminotransferase, Aspartate Aminotransferase, Chloride, Protein, Alkaline Phosphatase, Bilirubin, Calcium, Creatinine, Glucose, Hematocrit, Hemoglobin, Potassium, Platelets, Erythrocytes, Sodium, and Leukocytes.

Item 20: The method of any of Items 1 to 19, wherein the health record associated with each timepoint in the sequence of timepoints include medical image data and/or electrogram data.

Item 21: The method of Item 20, wherein the medical image data includes a metric quantifying a severity of the disease portrayed in one or more medical images and/or electrograms.

Item 22: The method of any of Items 1 to 21, further comprising: determining that the longitudinal data includes a missing value for a longitudinal variable at a first timepoint; and replacing the missing value with (i) a first value of the longitudinal variable from a second timepoint preceding the first timepoint, (ii) a second value of the longitudinal variable from a third timepoint following the first timepoint, or (iii) a third value determined based on the first value and the second value.

Item 23: The method of any of Items 1 to 22, wherein the disease is coronavirus disease (COVID-19), Alzheimer's disease, or age-related macular degeneration.

Item 24: The method of any of Items 1 to 23, wherein the clinical outcome of the disease includes a probability associated with one or more of cure, worsening, and mortality.

Item 25: The method of any of Items 1 to 24, wherein the health record associated with each timepoint in the sequence of timepoints is an electronic health record (EHR).

Item 26: A system, comprising: at least one data processor; and at least one memory storing instructions, which when executed by the at least one data processor, result in operations comprising the method of any of Items 1 to 25.

Item 27: A non-transitory computer readable medium storing instructions, which when executed by at least one data processor, result in operations comprising the method of any of Items 1 to 25.

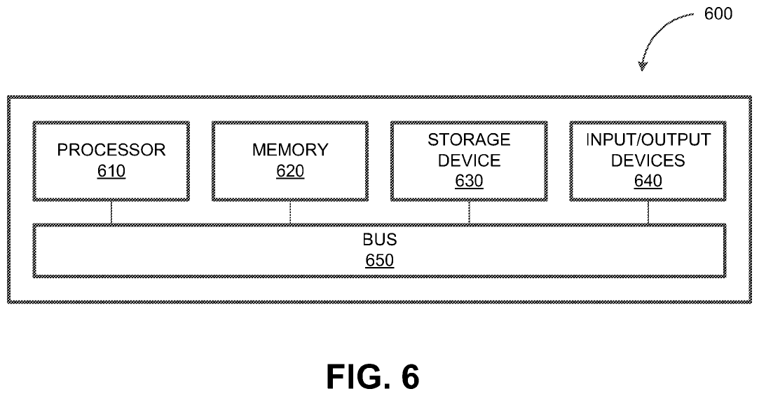

FIG. 6 depicts a block diagram illustrating an example of a computing system 600 consistent with implementations of the current subject matter. Referring to FIGS. 1-6, the computing system 600 can be used to implement the database management system 110 and/or any components therein.

As shown in FIG. 6, the computing system 600 can include a processor 610, a memory 620, a storage device 630, and an input/output device 640. The processor 610, the memory 620, the storage device 630, and the input/output device 640 can be interconnected via a system bus 650. The processor 610 is capable of processing instructions for execution within the computing system 600. Such executed instructions can implement one or more components of, for example, the database management system 110. In some example embodiments, the processor 610 can be a single-threaded processor. Alternately, the processor 610 can be a multi-threaded processor. The processor 610 is capable of processing instructions stored in the memory 620 and/or on the storage device 630 to display graphical information for a user interface provided via the input/output device 640.

The memory 620 is a computer readable medium such as volatile or non-volatile that stores information within the computing system 600. The memory 620 can store data structures representing configuration object databases, for example. The storage device 630 is capable of providing persistent storage for the computing system 600. The storage device 630 can be a solid state drive, a floppy disk device, a hard disk device, an optical disk device, or a tape device, or other suitable persistent storage means. The input/output device 640 provides input/output operations for the computing system 600. In some example embodiments, the input/output device 640 includes a keyboard and/or pointing device. In various implementations, the input/output device 640 includes a display unit for displaying graphical user interfaces.

According to some example embodiments, the input/output device 640 can provide input/output operations for a network device. For example, the input/output device 640 can include Ethernet ports or other networking ports to communicate with one or more wired and/or wireless networks (e.g., a local area network (LAN), a wide area network (WAN), the Internet).

In some example embodiments, the computing system 600 can be used to execute various interactive computer software applications that can be used for organization, analysis and/or storage of data in various formats. Alternatively, the computing system 600 can be used to execute any type of software applications. These applications can be used to perform various functionalities, e.g., planning functionalities (e.g., generating, managing, editing of spreadsheet documents, word processing documents, and/or any other objects, etc.), computing functionalities, communications functionalities, etc. The applications can include various add-in functionalities or can be standalone computing products and/or functionalities. Upon activation within the applications, the functionalities can be used to generate the user interface provided via the input/output device 640. The user interface can be generated and presented to a user by the computing system 600 (e.g., on a computer screen monitor, etc.).

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random query memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, recurrent provided to the user can be any form of sensory recurrent, such as for example visual recurrent, auditory recurrent, or tactile recurrent; and input from the user may be received in any form, including acoustic, speech, or tactile input. Other possible input devices include touch screens or other touch-sensitive devices such as single or multi-point resistive or capacitive track pads, voice recognition hardware and software, optical scanners, optical pointers, digital image capture devices and associated interpretation software, and the like.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

1. A system, comprising:
at least one data processor; and
at least one memory storing instructions, which when executed by the at least one data processor, result in operations comprising:
training a disease prognosis model to determine, based at least on longitudinal data, a clinical outcome of a disease, the longitudinal data including a health record for each timepoint in a sequence of timepoints, the training of the disease prognosis model includes training a recurrent neural network, a spacetime attention, and a feedforward neural network, the recurrent neural network being trained to extract, from each health record, a feature set representative of one or more local dependencies present within the health record, the spacetime attention being trained to determine an importance of each feature in the feature set at each timepoint in the sequence of timepoints, and the feedforward neural network being trained to determine, based at least on the importance of each feature in the feature set at each time point in the sequence of timepoints, the clinical outcome of the disease; and
applying the trained disease prognosis model to determine, based at least on a first health record from a first timepoint and a second health record from a second timepoint, the clinical outcome of the disease for a patient associated with the first health record and the second health record.

2. The system of claim 1, wherein the recurrent neural network is a bi-directional recurrent neural network (RNN), a long short-term memory (LSTM) network, a local long short-term memory (LSTM) network with a given window size for timepoints, or a gated recursive unit (GRU) network.

3. The system of claim 1, wherein the feedforward neural network is a multi-layer perceptron model.

4. The system of claim 1, wherein the trained disease prognosis model determines the clinical outcome of the disease for the patient associated with the first health record and the second health record by at least
applying the trained recurrent neural network to extract, from the first health record, a first set of feature values for a hidden feature set representative of a first set of local dependencies present within the first health record, and
applying the trained recurrent neural network to extract, from the second health record, a second set of feature values for the hidden feature set representative of a second set of local dependencies present within the second health record.

5. The system of claim 4, wherein the trained recurrent neural network outputs, for ingestion by the trained spacetime attention, a feature map comprising the first set of feature values from the first timepoint and the second set of feature values from the second timepoint.

6. The system of claim 4, wherein the trained disease prognosis model further determines the clinical outcome of the disease for the patient associated with the first health record and the second health record by at least applying the trained spacetime attention to determine, based at least on a feature map including the first set of feature values and the second set of feature values, the importance of each feature in the hidden feature set at each of the first timepoint and the second timepoint.

7. The system of claim 6, wherein the trained spacetime attention includes one or more two-dimensional convolutional layers trained to determine the importance of each feature in the hidden feature set across a time dimension and a feature dimension.

8. The system of claim 7, wherein the one or more two-dimensional convolutional layers include 1×1 convolutional filters configured to perform a joint weighting of the importance of each feature in the hidden feature set across the time dimension and the feature dimension.

9. The system of claim 6, wherein the trained spacetime attention determines, for a first feature from the hidden feature set, a first importance of the first feature at the first timepoint and a second importance of the first feature at the second timepoint.

10. The system of claim 9, wherein the trained spacetime attention further determines, for a second feature from the hidden feature set, a third importance of the second feature at the first timepoint and a fourth importance of the second feature at the second timepoint.

11. The system of claim 6, wherein the trained disease prognosis model further determines the clinical outcome of the disease for the patient associated with the first health record and the second health record by at least applying the trained feedforward neural network to determine, based at least on the importance of each feature in the hidden feature set at each of the first timepoint and the second timepoint, the clinical outcome of the disease for the patient.

12. The system of claim 1, wherein the disease prognosis model is further trained to determine the clinical outcome of the disease based on non-longitudinal data, wherein the non-longitudinal data is static across the sequence of timepoints, and wherein the non-longitudinal data is concatenated with the health record associated with each timepoint in the sequence of timepoints.

13. The system of claim 12, further comprising:
identifying one or more missing values for a non-longitudinal variable comprising the non-longitudinal data; and
replacing the one or more missing values with a mean value of the non-longitudinal variable observed in an available dataset.

14. The system of claim 12, wherein the non-longitudinal data includes medical image data and/or electrogram data corresponding to a metric quantifying a severity of the disease portrayed in one or more medical images and/or electrograms.

15. The system of claim 1, wherein the health record associated with each timepoint in the sequence of timepoints includes a value for each of a plurality of vital sign statistics.

16. The system of claim 1, wherein the health record associated with each timepoint in the sequence of timepoints includes a value for each of a plurality of laboratory test variables.

17. The system of claim 1, wherein the health record associated with each timepoint in the sequence of timepoints include medical image data and/or electrogram data, and wherein the medical image data includes a metric quantifying a severity of the disease portrayed in one or more medical images and/or electrograms.

18. The system of claim 1, further comprising:
determining that the longitudinal data includes a missing value for a longitudinal variable at a first timepoint; and
replacing the missing value with (i) a first value of the longitudinal variable from a second timepoint preceding the first timepoint, (ii) a second value of the longitudinal variable from a third timepoint following the first timepoint, or (iii) a third value determined based on the first value and the second value.

19. The system of claim 1, wherein the disease is coronavirus disease (COVID-19), Alzheimer's disease, or age-related macular degeneration.

20. The system of claim 1, wherein the clinical outcome of the disease includes a probability associated with one or more of cure, worsening, and mortality.

21. A computer-implemented method, comprising:
training a disease prognosis model to determine, based at least on longitudinal data, a clinical outcome of a disease, the longitudinal data including a health record for each timepoint in a sequence of timepoints, the training of the disease prognosis model includes training a recurrent neural network, a spacetime attention, and a feedforward neural network, the recurrent neural network being trained to extract, from each health record, a feature set representative of one or more local dependencies present within the health record, the spacetime attention being trained to determine an importance of each feature in the feature set at each timepoint in the sequence of timepoints, and the feedforward neural network being trained to determine, based at least on the importance of each feature in the feature set at each time point in the sequence of timepoints, the clinical outcome of the disease; and
applying the trained disease prognosis model to determine, based at least on a first health record from a first timepoint and a second health record from a second timepoint, the clinical outcome of the disease for a patient associated with the first health record and the second health record.

22. A non-transitory computer readable medium storing instructions, which when executed by at least one data processor, result in operations comprising:
training a disease prognosis model to determine, based at least on longitudinal data, a clinical outcome of a disease, the longitudinal data including a health record for each timepoint in a sequence of timepoints, the training of the disease prognosis model includes training a recurrent neural network, a spacetime attention, and a feedforward neural network, the recurrent neural network being trained to extract, from each health record, a feature set representative of one or more local dependencies present within the health record, the spacetime attention being trained to determine an importance of each feature in the feature set at each timepoint in the sequence of timepoints, and the feedforward neural network being trained to determine, based at least on the importance of each feature in the feature set at each time point in the sequence of timepoints, the clinical outcome of the disease; and
applying the trained disease prognosis model to determine, based at least on a first health record from a first timepoint and a second health record from a second timepoint, the clinical outcome of the disease for a patient associated with the first health record and the second health record.
